(19) United States
(12) Patent Application Publication
(10) Pub. No.: US 2024/0065758 A1
(43) Pub. Date: Feb. 29, 2024

(54) APPARATUS WITH COLLABORATIVE OPERATION BETWEEN HIGH-VOLTAGE PULSE FIELD ABLATION AND ELECTROPHYSIOLOGICAL RECORDING SYSTEM

(71) Applicant: SHANGHAI SHINEYO MEDICAL (GROUP) CO., LTD., Shanghai (CN)

(72) Inventors: Shuguo CHEN, Shanghai (CN); Juanfeng ZHANG, Shanghai (CN); Yungang ZHU, Shanghai (CN); Wenhai CHEN, Shanghai (CN)

(21) Appl. No.: 18/501,680

(22) Filed: Nov. 3, 2023

Related U.S. Application Data
(63) Continuation-in-part of application No. PCT/CN2022/088712, filed on Apr. 24, 2022.

(30) Foreign Application Priority Data
May 6, 2021 (CN) 202110490410.9

(51) Int. Cl. A61B 18/14

(52) U.S. Cl. CPC A61B 18/1492; A61B 2018/00577

(57) ABSTRACT

An apparatus with collaborative operation between high-voltage pulse field ablation and an electrophysiological recording system pertains to the field of medical devices and includes a high-voltage power supply, an energy storage capacitor, a discharge circuit, a high-frequency high-voltage pulse signal generation circuit, a switch array circuit and a control system. The energy storage capacitor is connected respectively to the high-voltage power supply, the discharge circuit and the high-frequency high-voltage pulse signal generation circuit. The switch array is connected respectively to the high-frequency high-voltage pulse signal generation circuit, a catheter and the electrophysiological recording system. The control system is connected respectively to the high-voltage power supply, the discharge circuit, the high-frequency high-voltage pulse signal generation circuit, the switch array circuit, a cardiac electrical signal monitor and a foot pedal switch.

### RELATED APPLICATIONS

This application is a continuation-in-part (CIP) application of PCT/CN2022/088712 filed on Apr. 24, 2022, which claims priority to Chinese Patent Application No. 202110490410.9 filed on May 6, 2021, the disclosures of which are incorporated herein in their entirety by reference.

### FIELD OF THE INVENTION

The present invention relates to the field of medical devices and, in particular, to an apparatus with collaborative operation between high-voltage pulse field ablation and an electrophysiological recording system.

### DESCRIPTION OF THE PRIOR ART

Atrial fibrillation (AF) is the most common cardiac arrhythmia with an incidence of approximately 2% and increasing with age. The most serious complication of AF is thromboembolism, a condition that may lead to stroke, myocardial infarction and the like. Stroke is a complication that is the most common cause of AF death.

AF therapies are divided into two categories: pharmacotherapeutic and non-pharmacotherapeutic. According to “Atrial Fibrillation: Current Understanding and Treatment Recommendations (2015)” published by the Cardiac Electrophysiology and Pacing Branch of the Chinese Medical Association, current pharmacotherapeutic options for AF primarily include ventricular rate control, restoration and maintenance of sinus rhythm and anti-thrombotic therapy. Other pharmacotherapeutic options include antiarrhythmic medication and anticoagulation. Antiarrhythmic medication attempts to prevent the occurrence of AF, control the rapid heart rate during AF, eliminate AF and maintain sinus rhythm, and often uses drugs such as propafenone, digoxin, Betaloc and Cordarone. Anticoagulation seeks to prevent the formation of atrial mural thrombi, which may detach and clog other organs, especially the brain. Warfarin is commonly used as an anticoagulant for this purpose.

Non-pharmacotherapeutic AF therapies include ablation, surgery and pacing, providing options for those who cannot benefit much from, or are not suitable for, medication. Successful ablation or surgical treatment can cure AF.

At present, catheter ablation has been recognized as an effective approach for the recovery and sinus rhythm maintenance of AF patients. Although most catheter ablation procedures utilize radio frequency (RF) energy, other forms of energy can also be used (including cryogenic, ultrasound, laser, etc.) However, ablation based on the conduction of heat/cold energy suffers from some limitations, because it may cause damage to the esophagus, the coronary arteries or the phrenic nerve in the neighborhood due to a lack of selectivity for tissue to be destroyed by ablation and reliance on the adhesion of the catheter to the tissue to be ablated. Consequently, certain complications may be reported in the perioperative period, and AF may recur in some patients for reasons relating to catheter adhesion and lesion depth. According to reports, a rate of recurrence after RF ablation is 20-40%, and that after cryogenic ablation is 10-30%.

In recent years, some attempts have been made in China and abroad to explore the application of pulsed electric fields (PEF) to cardiac ablation, and some promising results have been reported. Different from traditional energy, PEF energy can create irreversible micropores in the membrane of a cell through instantaneous discharge, eventually causing apoptosis of the cell and achieving non-thermal ablation, also known as irreversible electroporation. Currently, electroporation ablation has been utilized as an effective approach to destroy malignant tumor tissue. Theoretically, PEF ablation is able to damage myocardial cells without heating the tissue and provide cell/tissue selectivity and hence protection to crucial structures around the ablated tissue.

Pulse ablation works by using short high-voltage DC pulses to create electrical fields of hundreds of volts within a few centimeters, which can damage and perforate the cell membrane. If the strength of the electric fields created around the cell membrane exceeds a threshold, the resulting pores will be irreversible and remain open, eventually inducing apoptosis or necrosis of the cell. Therefore, pulse ablation is a non-thermal biological ablation technique, which, differing from RF, cryogenic, microwave and ultrasound ablation, can effectively avoid causing damage to blood vessels, nerves or the esophagus.

High-voltage pulse field ablation is a promising technique for future development of medical ablation, and real-time monitoring of ablation efficacy of high-voltage electric pulses represents not only a trend of future development but also a challenge requiring urgent resolution. The currently most common practice of electrophysiological ablation involves using an electrophysiological recording system to display electrophysiological signals collected by a mapping catheter from the heart during an electrophysiological ablation procedure for real-time observation and to store the signals. However, this practice suffers from a number of disadvantages as follows. The mapping catheter is not able to measure cardiac electrical potential signals from the actual tissue site where the ablation occurs, and the measurement location is somewhat offset from the actual tissue site. High-voltage electric pulse signals may enter the electrophysiological recording system. When this happens, not only the cardiac electrical potential signals may be interfered with and thus could not correctly reflect the real ablation efficacy, but the electrophysiological recording system may also be damaged.

Therefore, those skilled in the art are directing their effort toward developing an apparatus with collaborative operation between high-voltage pulse field ablation and an electrophysiological recording system, which is capable of real-time observation, acquisition, di splaying and storage of electrophysiological signals from heart during an electrophysiological ablation procedure without relevant signals being interfered with or obscured by high-voltage electric pulse signals and thereby providing correct and accurate measurements.

### SUMMARY OF THE INVENTION

In view of the above-described disadvantages of the prior art, the problem sought to be solved by the present invention is how to design and develop an apparatus with collaborative operation between high-voltage pulse field ablation and an electrophysiological recording system, which can overcome the problems of a measurement location deviation of a mapping catheter, interference of high-voltage pulse signals with an electrophysiological recording system, and possible damage caused by the signals to the recording system.

To this end, the present invention provides an apparatus with collaborative operation between high-voltage pulse field ablation and an electrophysiological recording system, comprising a high-voltage power supply, an energy storage capacitor, a discharge circuit, a high-frequency high-voltage pulse signal generation circuit, a switch array circuit and a control system, wherein the energy storage capacitor is connected respectively to the high-voltage power supply, the discharge circuit and the high-frequency high-voltage pulse signal generation circuit; the switch array is connected respectively to the high-frequency high-voltage pulse signal generation circuit, a catheter and the electrophysiological recording system; and the control system is connected respectively to the high-voltage power supply, the discharge circuit, the high-frequency high-voltage pulse signal generation circuit, the switch array circuit, a cardiac electrical signal monitor and a foot pedal switch.

Further, a positive pole of the high-voltage power supply may be connected to a first terminal of the energy storage capacitor, and a negative pole of the high-voltage power supply may be connected to a second terminal of the energy storage capacitor, wherein:
a first terminal of the discharge circuit is connected to the first terminal of the energy storage capacitor, and a second terminal of the discharge circuit is connected to the second terminal of the energy storage capacitor;
a first input terminal of the high-frequency high-voltage pulse signal generation circuit is connected to the first terminal of the energy storage capacitor, a second input terminal of the high-frequency high-voltage pulse signal generation circuit is connected to the second terminal of the energy storage capacitor, and an output terminal of the high-frequency high-voltage pulse signal generation circuit is connected to an input terminal of the switch array circuit;
a first output terminal of the switch array circuit is connected to the catheter, and a second output terminal of the switch array circuit is connected to the electrophysiological recording system;
the control system is connected to the high-voltage power supply by an RS232 or RS485 connection and used to control an output value of an output DC voltage of the high-voltage power supply and feed the output value of the DC voltage back to the control system;
the control system is connected to the discharge circuit and control the discharge circuit to discharge energy stored in the energy storage capacitor through a discharge control signal;
the control system is connected to the high-frequency high-voltage pulse signal generation circuit and is used to control output and deactivation of a pulse voltage and collect signals of the pulse voltage and a pulse current;
the control system is connected to the switch array circuit and controls the switch array circuit to operate as desired through a control signal;
the control system is connected to the cardiac electrical signal monitoring device and used to receive a trigger signal, which is sent by the cardiac electrical signal monitoring device after it detects an R wave; and
the control system is connected to the foot pedal switch and used to detect a signal from the foot pedal switch and thereby control output of high-voltage electric pulses.

Further, the high-frequency high-voltage pulse signal generation circuit may comprise two DC high-voltage source interfaces, four pulse-width modulated drive signal interfaces, four switching units and two pulse output interfaces, the two DC high-voltage source interfaces being respectively a positive power supply pole interface and a negative power supply pole interface, the four pulse-width modulated drive signal interfaces being respectively a first drive signal interface, a second drive signal interface, a third drive signal interface and a fourth drive signal interface, the four switching units being respectively a first switching unit, a second switching unit, a third switching unit and a fourth switching unit, the two pulse output interfaces being respectively a first pulse output interface and a second pulse output interface.

Further, the four switching units may be connected in series. One terminal of the first switching unit may be connected to the positive power supply pole interface, and another terminal of the first switching unit may be connected to one terminal of the fourth switching unit and the first pulse output interface. One terminal of the second switching unit may be connected to the positive power supply pole interface, and another terminal of the second switching unit may be connected to one terminal of the third switching unit and the second pulse output interface. Another terminal of the third switching unit may be connected to the negative power supply pole interface, and another terminal of the fourth switching unit may be connected to the negative power supply pole interface.

Further, each of the switching units may comprise an equal number of switching elements.

Further, each of the switching units may comprise one switching element.

Further, all the switching elements may be implemented as IGBTs, or as high-voltage MOS transistors.

Further, each switching element may be implemented as an IGBT or high-voltage MOS transistor with identical parameters.

Further, the IGBT may be an n-channel IGBT, or the high-voltage MOS transistor may be an n-channel SiC MOS transistor.

Further, a gate of the IGBT may serve as a control terminal of the switching unit, wherein an emitter of the IGBT of the first switching unit is connected to a collector of the IGBT of the fourth switching unit, and an emitter of the IGBT of the second switching unit is connected to a collector of the IGBT of the third switching unit. Alternatively, a gate of the high-voltage MOS transistor may serve as a control terminal of the switching unit, wherein a source of the high-voltage MOS transistor of the first switching unit is connected to a drain of the high-voltage MOS transistor of the fourth switching unit, and a source of the high-voltage MOS transistor of the second switching unit is connected to a drain of the high-voltage MOS transistor of the third switching unit.

Further, the first drive signal interface may be connected to the control terminal of the first switching unit, the second drive signal interface may be connected to a control terminal of the second switching unit, the third drive signal interface may be connected to a control terminal of the third switching unit, and the fourth drive signal interface may be connected to a control terminal of the fourth switching unit.

Further, the switch array circuit may comprise N high-voltage relay banks, where N≥2, wherein the N high-voltage relay banks are connected to respective N channels of the electrophysiological recording system; the N high-voltage relay banks are connected to respective N electrodes of the catheter; each of the high-voltage relay banks is made up of two series-connected high-voltage relays, which are respectively a first high-voltage relay and a second high-voltage relay; and the high-voltage relays are implemented as SPDT high-voltage vacuum relays with identical parameters.

Further, a normally closed contact of the first high-voltage relay may be connected to the first pulse output interface, wherein a normally open contact of the first high-voltage relay is connected to the second pulse output interface; a common terminal of the first high-voltage relay is connected to a normally open contact of the second high-voltage relay; a normally closed contact of the second high-voltage relay is connected to one channel of the electrophysiological recording system; a common terminal of the second high-voltage relay is connected to one electrode of the catheter; and the control system is connected to the switch array circuit and used to control activation and deactivation of the high-voltage relays.

Compared with the prior art, the present invention has the obvious substantive features and prominent advantages as follows:
1. The catheter can function either as a high-voltage pulse field ablation catheter, or as a mapping catheter.
2. During an electrophysiological ablation procedure, real-time collection, displaying and storage of cardiac electrical signals from the same tissue ablation location can be achieved.
3. Real-time observation, collection, displaying and storage of cardiac electrical signals are made possible during an electrophysiological ablation procedure.
4. During an electrophysiological ablation procedure, real-time observation, collection, displaying and storage of cardiac electrical signals can be carried out without relevant signals being interfered with or obscured by high-voltage electric pulse signals, thereby providing correct and accurate measurements.

Below, the concept, structural details and resulting technical effects of the present invention will be further described with reference to the accompanying drawings to provide a full understanding of the objects, features and effects of the invention.

### BRIEF DESCRIPTION OF THE DRAWINGS

FIG. 1 is a schematic diagram showing the structure of a preferred embodiment of the present invention.

FIG. 2 is a schematic diagram of a high-frequency high-voltage pulse signal generation circuit according to a preferred embodiment of the present invention.

FIG. 3 is a schematic diagram of a switch array circuit according to a preferred embodiment of the present invention.

In these figures, 11 denotes a high-voltage power supply; 12, an energy storage capacitor; 13, a discharge circuit; 14, a high-frequency high-voltage pulse signal generation circuit; 15, a switch array circuit; 16, a catheter; 17, a control system; 18, an electrophysiological recording system; 19, foot pedal switch; and 20, cardiac electrical signal monitoring device.

### DETAILED DESCRIPTION OF THE PREFERRED EMBODIMENTS

A few preferred embodiments of the present invention will be described more fully hereinafter with reference to the accompanying drawings so that technical contents thereof will become more apparent and easier to understand. The invention can be embodied in various different forms and its scope of protection is in no way limited to the embodiments discussed herein.

Throughout the figures, parts of the same structures are marked with the same reference numerals, and like elements with similar structures or functions are marked with like reference numerals. The dimensions and thickness of each component in the accompanying drawings are arbitrarily shown, and the present application is not limited to any particular dimensions and thickness of each component. Certain parts may be shown somewhat exaggerated in thickness in the interest of clarity.

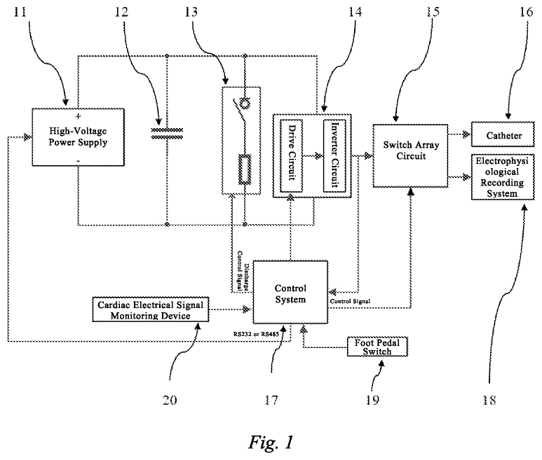

As shown in FIG. 1, an apparatus with collaborative operation between high-voltage pulse field ablation and an electrophysiological recording system includes a high-voltage power supply 11, an energy storage capacitor 12, a discharge circuit 13, a high-frequency high-voltage pulse signal generation circuit 14, a switch array circuit 15 and a control system 17. A positive pole of the high-voltage power supply 11 is connected to a first terminal of the energy storage capacitor 12, and a negative pole of the high-voltage power supply 11 is connected to a second terminal of the energy storage capacitor 12. A first terminal of the discharge circuit 13 is connected to the first terminal of the energy storage capacitor 12, and a second terminal of the discharge circuit 13 is connected to the second terminal of the energy storage capacitor 12. A first input terminal of the high-frequency high-voltage pulse signal generation circuit 14 is connected to the first terminal of the energy storage capacitor 12, and a second input terminal of the high-frequency high-voltage pulse signal generation circuit 14 is connected to the second terminal of the energy storage capacitor 12. An output terminal of the high-frequency high-voltage pulse signal generation circuit 14 is connected to an input terminal of the switch array circuit 15. A first output terminal of the switch array circuit 15 is connected to the catheter 16, and a second output terminal of the switch array circuit 15 is connected to the electrophysiological recording system 18.

The control system 17 is connected to the high-voltage power supply 11 by an RS232 or RS485 connection. In this way, the value of a DC voltage output from the high-voltage power supply 11 can be controlled and fed back to the control system. The control system 17 is connected to the discharge circuit 13 and controls the discharge circuit 13 to discharge energy stored in the energy storage capacitor 12 through a discharge control signal. The control system 17 is connected to the high-frequency high-voltage pulse signal generation circuit 14, in order to control the output and deactivation of the pulse voltage and collect signals of the pulse voltage and a pulse current. The control system 17 is connected to the cardiac electrical signal monitoring device 20, in order to receive a trigger signal, which is sent by the cardiac electrical signal monitoring device 20 after it detects an R wave. The control system 17 is connected to the foot pedal switch 19, in order to detect a signal from the foot pedal switch 19 and thereby control the output of high-voltage electric pulses. The control system 17 is connected to the switch array circuit 15. The control system uses a control signal to control the switch array circuit to operation as required. The control system 17 may be implemented as a system known in the art, and examples of it may include, but are not limited to, PCs and embedded systems. The catheter 16 may be selected as a catheter known in the art for use in pulse ablation. The present invention is not limited to any particular structure of the catheter 16. The foot pedal switch 19 is a switch, which can be stepped on or tapped on with a foot to control the activation and deactivation of a circuit. It may be implemented as a foot pedal switch known in the art.

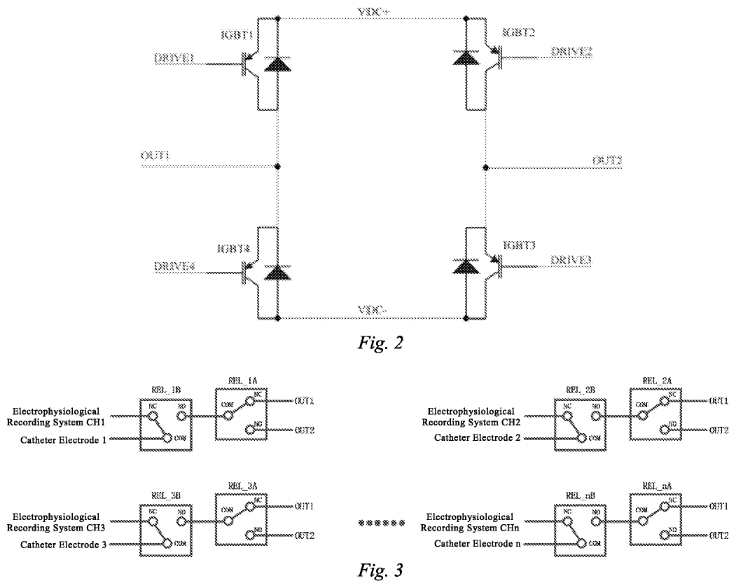

FIG. 2 is a schematic diagram of the high-frequency high-voltage pulse signal generation circuit 14. As shown, it includes: two DC high-voltage source interfaces VDC+, VDC−; four pulse-width modulated drive signal interfaces DRIVE1, DRIVE2, DRIVE3, DRIVE4; four switching units, namely, a first switching unit, a second switching unit, a third switching unit and a fourth switching unit; and two pulse output interfaces OUT1, OUT2. The four switching units are connected in series. A terminal of the first switching unit is connected to the interface VDC+, and another terminal of the first switching unit is connected to a terminal of the fourth switching unit and OUT1. A terminal of the second switching unit is connected to the interface VDC+, and another terminal of the second switching unit is connected to a terminal of the third switching unit and OUT2. Another terminal of the third switching unit is connected to the interface VDC−, and another terminal of the fourth switching unit is connected to the interface VDC−.

In this embodiment, the switching units are implemented as IGBTs with identical parameters. Moreover, the IGBTs are n-channel IGBTs each having a gate serving as a control terminal of the switching unit. An emitter of IGBT1 is connected to a collector of IGBT4, and an emitter of IGBT2 is connected to a collector of IGBT3.

DRIVE1 is connected to the control terminal of the first switching unit. DRIVE2 is connected to the control terminal of the second switching unit. DRIVE3 is connected to the control terminal of the third switching unit. DRIVE4 is connected to the control terminal of the fourth switching unit.

FIG. 3 is a schematic diagram of the switch array circuit 15 according to this embodiment. As shown, it includes N high-voltage relay banks, where N≥2. Each high-voltage relay bank has four interfaces connected respectively to OUT1, OUT2 of the high-frequency high-voltage pulse signal generation circuit 14, a channel of the electrophysiological recording system 18 and an electrode of the catheter 16. The high-voltage relays are implemented as SPDT high-voltage vacuum relays with identical parameters.

In detail, one of the high-voltage relay banks is made up of a high-voltage relay REL_1A and a high-voltage relay REL_1B. A COM terminal of the high-voltage relay REL_1A is connected to a NO terminal of the high-voltage relay REL_1B. A NC terminal of the high-voltage relay REL_1A is connected to OUT1 of the high-frequency high-voltage pulse signal generation circuit 14, and a NO terminal of the high-voltage relay REL_1A is connected to OUT2 of the high-frequency high-voltage pulse signal generation circuit 14. ANC terminal of the high-voltage relay REL_1B is connected to an electrocardiographic channel CH1 of the electrophysiological recording system 18, and a COM terminal of the high-voltage relay REL_1B is connected to an electrode 1 of the catheter 16.

One of the high-voltage relay banks is made up of a high-voltage relay REL_2A and a high-voltage relay REL_2B. A COM terminal of the high-voltage relay REL_2A is connected to a NO terminal of the high-voltage relay REL_2B. A NC terminal of the high-voltage relay REL_2A is connected to OUT1 of the high-frequency high-voltage pulse signal generation circuit 14, and a NO terminal of the high-voltage relay REL_2A is connected to OUT2 of the high-frequency high-voltage pulse signal generation circuit 14. A NC terminal of the high-voltage relay REL_2B is connected to an electrocardiographic channel CH2 of the electrophysiological recording system 18, and a COM terminal of the high-voltage relay REL_2B is connected to an electrode 2 of the catheter 16.

One of the high-voltage relay banks is made up of a high-voltage relay REL_3A and a high-voltage relay REL_3B. A COM terminal of the high-voltage relay REL_3A is connected to a NO terminal of the high-voltage relay REL_3B. A NC terminal of the high-voltage relay REL_3A is connected to OUT1 of the high-frequency high-voltage pulse signal generation circuit 14, and a NO terminal of the high-voltage relay REL_3A is connected to OUT2 of the high-frequency high-voltage pulse signal generation circuit 14. A NC terminal of the high-voltage relay REL_3B is connected to an electrocardiographic channel CH3 of the electrophysiological recording system 18, and a COM terminal of the high-voltage relay REL_3B is connected to an electrode 3 of the catheter 16.

By analogy, one of the high-voltage relay banks is made up of a high-voltage relay REL_nA and a high-voltage relay REL_nB, where n≥2. A COM terminal of the high-voltage relay REL_nA is connected to a NO terminal of the high-voltage relay REL_nB. A NC terminal of the high-voltage relay REL_nA is connected to OUT1 of the high-frequency high-voltage pulse signal generation circuit 14, and a NO terminal of the high-voltage relay REL_nA is connected to OUT2 of the high-frequency high-voltage pulse signal generation circuit 14. ANC terminal of the high-voltage relay REL_nB is connected to an electrocardiographic channel CHn of the electrophysiological recording system 18, and a COM terminal of the high-voltage relay REL_1B is connected to an electrode n of the catheter 16.

The apparatus of this embodiment enables the catheter to function as both an ablation catheter and a mapping catheter. Specifically, during a high-voltage pulse field ablation procedure, the catheter is used to perform pulse ablation as a pulse ablation catheter. At the same time, the electrophysiological recording system 18 does not collect, display or store cardiac electrical signals through the catheter. After the pulse ablation procedures is completed, the catheter automatically switches to be connected to the electrophysiological recording system 18 and serves as a mapping catheter to enable real-time collection, displaying and storage of cardiac electrical signals. This exempts an operator from manual replacement or switching of ablation and mapping catheters during the procedure.

The switching of the catheter 16 switches will be described in detail below.

In one implementation of this embodiment, as an example, the electrodes 1, 3, 5, . . . , n1 of the catheter 16 are connected to OUT1, and the electrodes 2, 4, 6, . . . , n2 of the catheter 16 are connected to OUT2 (n1 is an odd number less than n, and n2 is an even number less than n). The NC terminals of all the high-voltage relays REL_1A, REL_2A, REL_3A, REL_nA are connected to the interface OUT1, and the NO terminals of all the high-voltage relays REL_1A, REL_2A, REL_3A, REL_nA are connected to the interface OUT2. The COM terminals of the high-voltage relays REL_1A, REL_2A, REL_3A, REL_nA are connected to the respective NO terminals of the high-voltage relays REL_1B, REL_2B, REL_3B, REL_nB. The NC terminals of the high-voltage relays REL_1B, REL_2B, REL_3B, REL_nB are connected to the respective electrocardiographic channels CH1, CH2, CH3, CHn of the electrophysiological recording system 18, where n is the maximum number of channels that the electrophysiological recording system 18 can support. The COM terminals of the high-voltage relays REL_1B, REL_2B, REL_3B, REL_nB are connected to the respective electrodes 1, 2, 3, . . . , n of the catheter. First of all, the electrophysiological recording system 18 performs pre-ablation real-time observation, collection, displaying and storage of cardiac electrophysiological signals through the catheter 16. Under the control of the control system 17, coils in the high-voltage relays REL_2A, REL_4A, REL_6A, . . . , REL_n2A are energized to produce attractive forces, which cause connection of the COM terminals thereof with the respective NO terminals. After that, when the cardiac electrical signal monitoring device 20 detects an R wave, it sends a trigger signal to the control system 17 after a delay period following the detection, which is as long as one cardiac cycle. This ensures that high-voltage electric pulses are output within a refractory period at the end of an interval equal to one cardiac cycle. In response to the trigger signal from the cardiac electrical signal monitoring device 20, the control system 17 then gets ready for output of high-voltage pulse signals and starts waiting for a signal from the foot pedal. Once the foot pedal switch 19 is pushed down with a foot, coils in the high-voltage relays REL_1B, REL_2B, REL_3B, . . . , REL_nB are energized to produce attractive forces, which cause connection of the COM terminals thereof with the respective NO terminals. As a result, the high-frequency high-voltage pulse signal generation circuit 14 outputs high-voltage pulse signals, which are transmitted through the switch array circuit 15 to the catheter 16 to effect tissue ablation. After the foot pedal switch 19 is released, the coils in the high-voltage relays REL_1B, REL_2B, REL_3B, . . . , REL_nB are unenergized, causing connection of the COM terminals thereof with the respective NC terminals. The electrophysiological recording system 18 performs post-ablation real-time observation, collection, displaying and storage of cardiac electrophysiological signals. When the foot pedal switch 19 is again pushed down, the coils in the high-voltage relays REL_1B, REL_2B, REL_3B, . . . , REL_nB will be energized to produce attractive forces, which cause connection of the COM terminals with the respective NO terminals. As a result, the high-frequency high-voltage pulse signal generation circuit 14 will output high-voltage pulse signals, which are transmitted through the switch array circuit 15 to the catheter 16 to effect tissue ablation. When the foot pedal switch 19 is released, the coils in the high-voltage relays REL_1B, REL_2B, REL_3B, . . . , REL_nB will be unenergized, causing connection of the COM terminals thereof with the respective NC terminals. The electrophysiological recording system 18 will then again perform post-ablation detection of cardiac electrical potential signals through the catheter 16. This process can be carried out for any desired times.

In another implementation of this embodiment, the electrophysiological recording system 18 first performs pre-ablation real-time observation, collection, di splaying and storage of cardiac electrical signals, and then under the control of the control system 17, the coils in the high-voltage relays REL_2A, REL_4A, REL_6A, . . . , REL_n2A are energized to produce attractive forces, which cause connection of the COM terminals thereof to the respective NO terminals. After that, when the cardiac electrical signal monitoring device 20 detects an R wave, it sends a trigger signal to the control system 17 after a delay period following the detection, which is as long as one cardiac cycle. This ensures that high-voltage electric pulses are output within a refractory period at the end of an interval equal to one cardiac cycle. In response to the trigger signal from the cardiac electrical signal monitoring device 20, the control system 17 then gets ready for output of high-voltage pulse signals and starts waiting for a signal from the foot pedal. Once the foot pedal switch 19 is pushed down with a foot, the coils in the high-voltage relays REL_1B, REL_2B, REL_3B, . . . , REL_nB are energized to produce attractive forces, which cause connection of the COM terminals thereof with the respective NO terminals. As a result, the high-frequency high-voltage pulse signal generation circuit 14 outputs high-voltage pulse signals, which are transmitted through the switch array circuit 15 to the catheter 16 to effect tissue ablation. Over said interval equal to one cardiac cycle, the coils in the high-voltage relays REL_1B, REL_2B, REL_3B, . . . , REL_nB are unenergized, causing connection of the COM terminals thereof with the respective NC terminals. The electrophysiological recording system 18 performs post-ablation real-time observation, collection, displaying and storage of cardiac electrophysiological signals. Upon an R wave signal being detected, the coils in the high-voltage relays REL_1B, REL_2B, REL_3B, . . . , REL_nB are energized to produce attractive forces, which cause connection of the COM terminals thereof to the respective NO terminals. The high-frequency high-voltage pulse signal generation circuit 14 outputs high-voltage pulse signals, which are transmitted through the switch array circuit 15 to the catheter 16 to effect tissue ablation. Over said interval equal to one cardiac cycle, the coils in the high-voltage relays REL_1B, REL_2B, REL_3B, . . . , REL_nB are unenergized, causing connection of the COM terminals thereof with the respective NC terminals. The electrophysiological recording system 18 performs post-ablation real-time observation, collection, displaying and storage of cardiac electrophysiological signals. This process can be carried out for any desired times.

In another embodiment, all the switching elements are implemented as high-voltage MOS transistors with identical parameters, which are n-channel SiC MOS transistors. Gates of the high-voltage MOS transistors serve as control terminals of the switching units. A source of the high-voltage MOS transistor serving as the first switching unit is connected to a drain of the high-voltage MOS transistor serving as the fourth switching unit. A source of the high-voltage MOS transistor serving as the second switching unit is connected to a drain of the high-voltage MOS transistor serving as the third switching unit.

Preferred specific embodiments of the present invention have been described in detail above. It is to be understood that, those of ordinary skill in the art can make various modifications and changes based on the concept of the present invention without exerting any creative effort. Accordingly, all the technical solutions that can be obtained by those skilled in the art by logical analysis, inference or limited experimentation in accordance with the concept of the present invention on the basis of the prior art are intended to fall within the protection scope as defined by the claims.

1. An apparatus with collaborative operation between high-voltage pulse field ablation and an electrophysiological recording system, comprising a high-voltage power supply, an energy storage capacitor, a discharge circuit, a high-frequency high-voltage pulse signal generation circuit, a switch array circuit and a control system, wherein the energy storage capacitor is connected respectively to the high-voltage power supply, the discharge circuit and the high-frequency high-voltage pulse signal generation circuit; the switch array is connected respectively to the high-frequency high-voltage pulse signal generation circuit, a catheter and the electrophysiological recording system; and the control system is connected respectively to the high-voltage power supply, the discharge circuit, the high-frequency high-voltage pulse signal generation circuit, the switch array circuit, a cardiac electrical signal monitor and a foot pedal switch.

2. The apparatus with collaborative operation between high-voltage pulse field ablation and an electrophysiological recording system of claim 1, wherein a positive pole of the high-voltage power supply is connected to a first terminal of the energy storage capacitor, and a negative pole of the high-voltage power supply is connected to a second terminal of the energy storage capacitor;
a first terminal of the discharge circuit is connected to the first terminal of the energy storage capacitor, and a second terminal of the discharge circuit is connected to the second terminal of the energy storage capacitor;
a first input terminal of the high-frequency high-voltage pulse signal generation circuit is connected to the first terminal of the energy storage capacitor, a second input terminal of the high-frequency high-voltage pulse signal generation circuit is connected to the second terminal of the energy storage capacitor, and an output terminal of the high-frequency high-voltage pulse signal generation circuit is connected to an input terminal of the switch array circuit;
a first output terminal of the switch array circuit is connected to the catheter, and a second output terminal of the switch array circuit is connected to the electrophysiological recording system;
the control system is connected to the high-voltage power supply by an RS232 or RS485 connection and used to control an output value of an output DC voltage of the high-voltage power supply and feed the output value of the DC voltage back to the control system;
the control system is connected to the discharge circuit and control the discharge circuit to discharge energy stored in the energy storage capacitor through a discharge control signal;
the control system is connected to the high-frequency high-voltage pulse signal generation circuit and is used to control output and deactivation of a pulse voltage and collect signals of the pulse voltage and a pulse current;
the control system is connected to the switch array circuit and controls the switch array circuit to operate as desired through a control signal;
the control system is connected to the cardiac electrical signal monitoring device and used to receive a trigger signal, which is sent by the cardiac electrical signal monitoring device after it detects an R wave; and
the control system is connected to the foot pedal switch and used to detect a signal from the foot pedal switch and thereby control output of high-voltage electric pulses.

3. The apparatus with collaborative operation between high-voltage pulse field ablation and an electrophysiological recording system of claim 2, wherein the high-frequency high-voltage pulse signal generation circuit comprises two DC high-voltage source interfaces, four pulse-width modulated drive signal interfaces, four switching units and two pulse output interfaces, the two DC high-voltage source interfaces being respectively a positive power supply pole interface and a negative power supply pole interface, the four pulse-width modulated drive signal interfaces being respectively a first drive signal interface, a second drive signal interface, a third drive signal interface and a fourth drive signal interface, the four switching units being respectively a first switching unit, a second switching unit, a third switching unit and a fourth switching unit, the two pulse output interfaces being respectively a first pulse output interface and a second pulse output interface, the four switching units connected in series, i.e., one terminal of the first switching unit connected to the positive power supply pole interface, another terminal of the first switching unit connected to one terminal of the fourth switching unit and the first pulse output interface, one terminal of the second switching unit connected to the positive power supply pole interface, another terminal of the second switching unit connected to one terminal of the third switching unit and the second pulse output interface, another terminal of the third switching unit connected to the negative power supply pole interface, another terminal of the fourth switching unit connected to the negative power supply pole interface, each of the switching units comprising a switching element.

4. The apparatus with collaborative operation between high-voltage pulse field ablation and an electrophysiological recording system of claim 3, wherein each switching element is an IGBT; a gate of the IGBT serves as a control terminal of the switching unit; an emitter of the IGBT of the first switching unit is connected to a collector of the IGBT of the fourth switching unit; and an emitter of the IGBT of the second switching unit is connected to a collector of the IGBT of the third switching unit.

5. The apparatus with collaborative operation between high-voltage pulse field ablation and an electrophysiological recording system of claim 3, wherein each switching element is a high-voltage MOS transistor; a gate of the high-voltage MOS transistor serves as a control terminal of the switching unit; a source of the high-voltage MOS transistor of the first switching unit is connected to a drain of the high-voltage MOS transistor of the fourth switching unit; and a source of the high-voltage MOS transistor of the second switching unit is connected to a drain of the high-voltage MOS transistor of the third switching unit.

6. The apparatus with collaborative operation between high-voltage pulse field ablation and an electrophysiological recording system of claim 4, wherein the IGBT is an n-channel IGBT.

7. The apparatus with collaborative operation between high-voltage pulse field ablation and an electrophysiological recording system of claim 5, wherein the high-voltage MOS transistor is an n-channel silicon carbide MOS transistor.

8. The apparatus with collaborative operation between high-voltage pulse field ablation and an electrophysiological recording system of claim 4, wherein the first drive signal interface is connected to a control terminal of the first switching unit; the second drive signal interface is connected to a control terminal of the second switching unit; the third drive signal interface is connected to a control terminal of the third switching unit; and the fourth drive signal interface is connected to a control terminal of the fourth switching unit.

9. The apparatus with collaborative operation between high-voltage pulse field ablation and an electrophysiological recording system of claim 8, wherein the switch array circuit comprises N high-voltage relay banks, where N≥2; the N high-voltage relay banks are connected to respective N channels of the electrophysiological recording system; the N high-voltage relay banks are connected to respective N electrodes of the catheter; each of the high-voltage relay banks is made up of two series-connected high-voltage relays, which are respectively a first high-voltage relay and a second high-voltage relay; and the high-voltage relays are implemented as SPDT high-voltage vacuum relays with identical parameters.

10. The apparatus with collaborative operation between high-voltage pulse field ablation and an electrophysiological recording system of claim 9, wherein a normally closed contact of the first high-voltage relay is connected to the first pulse output interface; a normally open contact of the first high-voltage relay is connected to the second pulse output interface; a common terminal of the first high-voltage relay is connected to a normally open contact of the second high-voltage relay; a normally closed contact of the second high-voltage relay is connected to one channel of the electrophysiological recording system; a common terminal of the second high-voltage relay is connected to one electrode of the catheter; and the control system is connected to the switch array circuit and used to control activation and deactivation of the high-voltage relays.

11. The apparatus with collaborative operation between high-voltage pulse field ablation and an electrophysiological recording system of claim 5, wherein the first drive signal interface is connected to a control terminal of the first switching unit; the second drive signal interface is connected to a control terminal of the second switching unit; the third drive signal interface is connected to a control terminal of the third switching unit; and the fourth drive signal interface is connected to a control terminal of the fourth switching unit.

12. The apparatus with collaborative operation between high-voltage pulse field ablation and an electrophysiological recording system of claim 11, wherein the switch array circuit comprises N high-voltage relay banks, where N≥2; the N high-voltage relay banks are connected to respective N channels of the electrophysiological recording system; the N high-voltage relay banks are connected to respective N electrodes of the catheter; each of the high-voltage relay banks is made up of two series-connected high-voltage relays, which are respectively a first high-voltage relay and a second high-voltage relay; and the high-voltage relays are implemented as SPDT high-voltage vacuum relays with identical parameters.

13. The apparatus with collaborative operation between high-voltage pulse field ablation and an electrophysiological recording system of claim 12, wherein a normally closed contact of the first high-voltage relay is connected to the first pulse output interface; a normally open contact of the first high-voltage relay is connected to the second pulse output interface; a common terminal of the first high-voltage relay is connected to a normally open contact of the second high-voltage relay; a normally closed contact of the second high-voltage relay is connected to one channel of the electrophysiological recording system; a common terminal of the second high-voltage relay is connected to one electrode of the catheter; and the control system is connected to the switch array circuit and used to control activation and deactivation of the high-voltage relays.
